(19) United States
(12) Patent Application Publication
(10) Pub. No.: US 2015/0208968 A1
(43) Pub. Date: Jul. 30, 2015

(54) SYSTEM AND METHOD FOR ASSESSMENT OF PATIENT HEALTH BASED ON RECOVERY RESPONSES FROM OXYGEN DESATURATION

(71) Applicant: KONINKLIJKE PHILIPS N.V., EINDHOVEN (NL)

(72) Inventors: Colleen Ennett, White Plains, NY (US); Stijn De Waele, Millwood, NY (US)

(73) Assignee: KONINKLIJKE PHILIPS N.V. a corporation

(21) Appl. No.: 14/424,055

(22) PCT Filed: Sep. 13, 2013
(86) PCT No.: PCT/IB2013/058536

§ 371 Date: Feb. 26, 2015

Related U.S. Application Data
(60) Provisional application No. 61/706,903, filed on Sep. 28, 2012.

(51) Int. Cl. A61B 5/1455; A61B 5/08; A61M 16/00

(52) U.S. Cl. CPC A61B 5/14552; A61M 16/0003; A61B 5/082; A61B 2505/03; A61B 2503/045; A61M 2240/00

(57) ABSTRACT

A system (10) for assessing the health of a patient includes one or more sensors (32) which generate medical data representing a patient's oxygen saturation and a health assessment system (12) which determines a recovery response from an oxygen desaturation event detected from the medical data and generates a health assessment based on the patient's recovery response. The recovery response being determined by at least one of determining a time period from a detection of the oxygenation desaturation event to the patient reaching predetermined oxygen saturation, determining an increase in the oxygen saturation after a fixed amount of time after detection of the oxygenation desaturation event, and determining a rate of an increase in the oxygen saturation.

The present application relates to assessing patient health. It finds particular application in conjunction with systems and methods for assessing the health of a patient and will be described with particular reference thereto. However, it is to be understood that it also finds application in other usage scenarios and is not necessarily limited to the aforementioned application.

In neonatal intensive care, a number of physiological parameters are monitored, including the oxygen saturation SpO₂ and the inspired oxygen FiO₂. Typically, infants have numerous short apnea events which are detected by monitoring these physiological parameters. Many of these apnea events go unnoticed or the infants recover before an intervention can be performed. Intervention may be as simple as touching the infant's foot or a gentle nudge.

Assessing adequacy of oxygenation and ventilation of infants receiving supplemental oxygen and ventilation support is generally done in an ad hoc manner. Oxygenation refers to the amount of oxygen going into the infant and making its way to the cells for oxygen exchange. Ventilation refers to the removal of carbon dioxide from the cells. There are many factors to consider when determining the oxygenation and ventilation status of an infant. Caregivers must have a clear understanding of the infants's health status to assess whether the current support is adequate, if there is a need to transition to another form of support, or if support is no longer needed.

Currently, caregivers integrate clinical data (arterial blood gases, ventilation settings, blood glucose, etc.) from multiple sources (laboratory information system, ventilator, pulse oximeter, etc.) to assess the adequacy of oxygenation and ventilation of the infant. Apnea events often go unnoticed because the caregiver is managing many patients at the same time, and consequently cannot monitor each neonate continuously.

The present application provides new and improved methods and systems which overcome the above-referenced problems and others.

In accordance with one aspect, a system for assessing the health of a patient is provided. The system including one or more sensors which generate medical data representing a patient's oxygen saturation and a health assessment system which determines a recovery response from an oxygen desaturation event detected from the medical data and generates a health assessment based on the patient's recovery response.

In accordance with another aspect, a system for assessing the health of a patient is provided. The system including one or more processors programmed to receive medical data representing a patient's oxygen saturation, detect an oxygen desaturation event from the medical data, determine a recovery response following the oxygen desaturation event, and display at least one of an indication of the recovery response and a health assessment from the recovery response.

In accordance with another aspect, a method for assessing the health of a patient is provided. The method including receiving medical data representing a patient's oxygen saturation, detecting an oxygen desaturation event from the medical data, determining a recovery response from the oxygen desaturation event, and displaying at least one of the determined recovery response and a health assessment based on the determined recovery response.

One advantage resides in the assessment of patient health based on recovery responses from oxygen desaturation.

Another advantage resides in analysis of patient support based on the recovery responses from oxygen desaturation.

Still further advantages of the present invention will be appreciated to those of ordinary skill in the art upon reading and understanding the following detailed description.

The invention may take form in various components and arrangements of components, and in various steps and arrangements of steps. The drawings are only for purposes of illustrating the preferred embodiments and are not to be construed as limiting the invention.

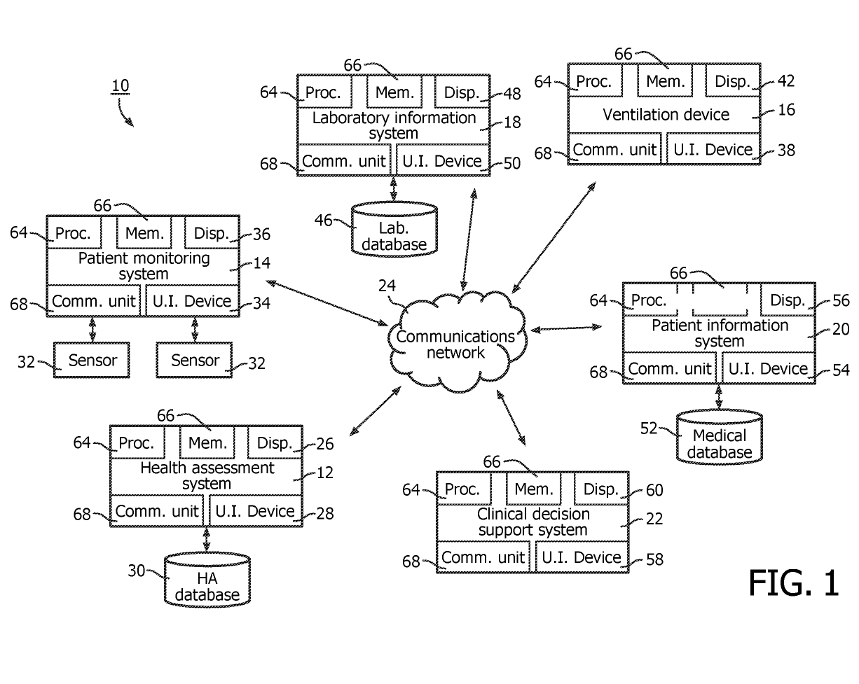

FIG. 1 is a block diagram of an IT infrastructure in accordance with the present application.

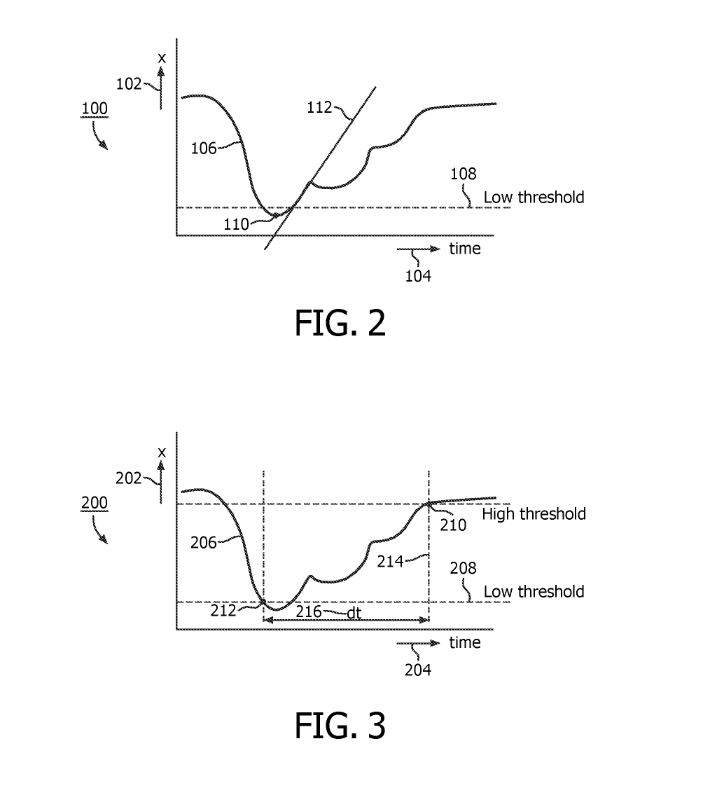

FIG. 2 is a quantification of recovery through time derivation/slope in accordance with the present application.

FIG. 3 is another quantification of recovery through time interval in accordance with the present application.

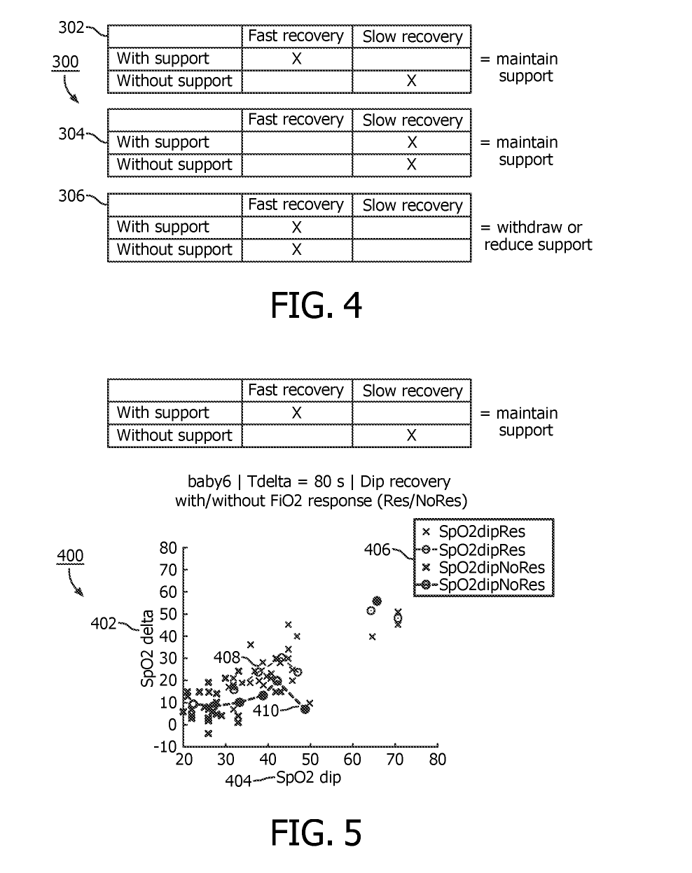

FIG. 4 illustrates several recovery and support scenarios and the recommendation for maintaining or withdrawing/reducing ventilation support in accordance with the present application.

FIG. 5 is an exemplary illustration of a response to a deteriorated health event in accordance with the present application.

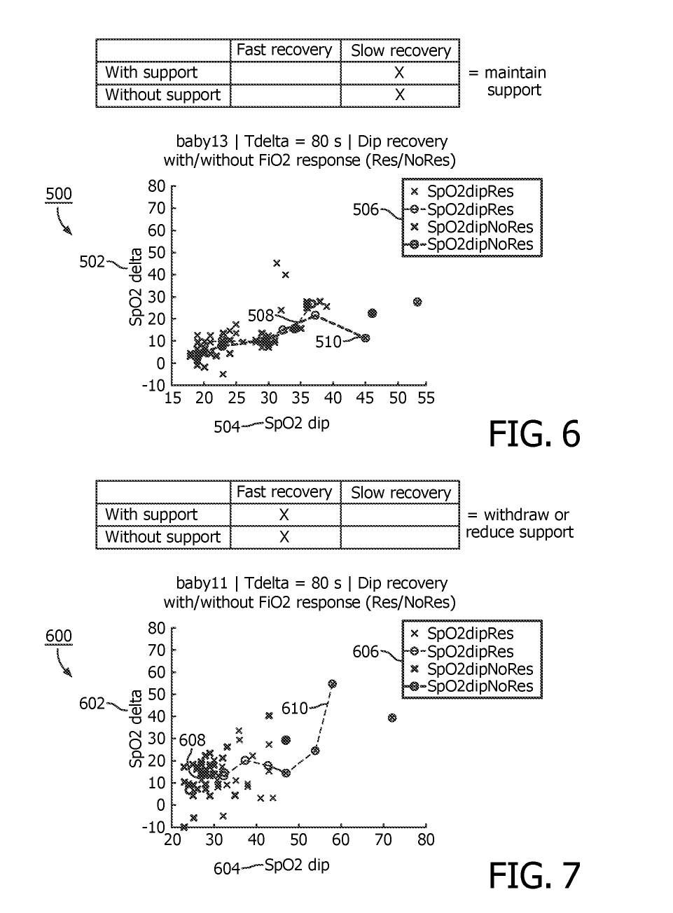

FIG. 6 is another exemplary illustration of a response to a deteriorated health event in accordance with the present application.

FIG. 7 is a further exemplary illustration of a response to a deteriorated health event in accordance with the present application.

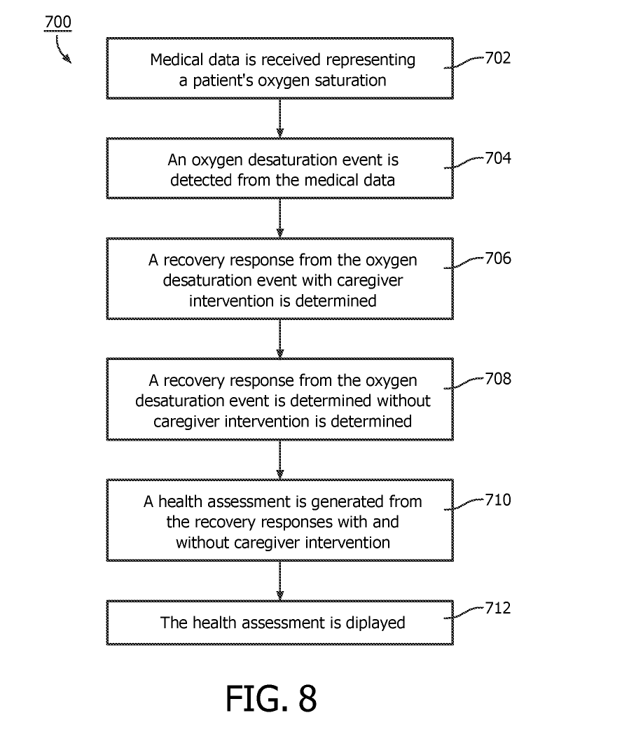

FIG. 8 is a flowchart diagram of a method for assessing patient health in accordance with the present application.

The present application utilizes oxygen saturation (SpO₂) waveforms and information about ventilation settings including fraction of inspired oxygen (FiO₂) to assess a patient's health status. SpO₂ waveform segments from oxygen desaturation events with SpO₂ measurements less than 85 percent are compared when there is caregiver intervention defined as an increase in FiO₂ and when there is no caregiver intervention. A patient's recovery response to an oxygen desaturation with and without caregiver intervention is an indicator of patient health. Besides application of FiO₂, the intervention can also consist of stimulating the baby to reinitiate breathing on its own. The presence of a caregiver with the neonate can for example be detected from the fact that the incubator was opened.

For example, infants in the Neonatal Intensive Care Unit (NICU) often experience oxygen desaturation events, where the blood oxygen level as measured by the pulse oximeter drops below 85 percent. In some cases, the infant is able to recover from the desaturation without intervention, and other times a caregiver intervenes with an increase in the fraction of inspired oxygen provided to the infant. The present application takes advantage of these events to assess the infant's health status based on response to the desaturation with and without intervention. Specifically, the present application compares desaturation events with and without intervention and utilizes this information to assess the infant's health status. Infants in better health recover faster from the desaturation than infants in worse health. It should also be appreciated that infants in better health typically recover from the desaturation event just as quickly with or without intervention, and infants in worse health typically recover from the desaturation just as poorly with or without intervention. The standard for assessing a patient's health is the duration of oxygen support required, because patients in better health have shorter durations of ventilation support than patients in worse health.

With reference to FIG. 1, a block diagram illustrates one embodiment of an information technology (IT) infrastructure 10 of a medical institution, such as a hospital. The IT infrastructure 10 suitably includes one or more health assessment systems 12, one or more patient monitoring systems 14, one or more ventilation devices 16, one or more laboratory information systems 18, a patient information system 20, a clinical decision support system 22, and the like, interconnected via a communications network 24. It is contemplated that the communications network 24 includes one or more of the Intranet, a local area network, a wide area network, a wireless network, a wired network, a cellular network, a data bus, and the like.

The health assessment system 12 assesses the health of the patients (not shown) cared for by the medical institution. The health assessment system 12 provides clinicians with an assessment of a patient's health and generates a health assessment indicating the patient's health status. The health assessment system 12 includes a display 26 such as a CRT display, a liquid crystal display, a light emitting diode display, to display the medical data and/or health assessment and a user input device 28 such as a keyboard and a mouse, for the caregiver to interpret the medical data and generate the health assessment. The health assessment system 12 acquires the medical data from the one or more patient monitoring systems 14, one or more ventilation devices 16, one or more laboratory information systems 18, a patient information system 20, and the like. After the medical data are acquired, the health assessment system 12 assesses the health of the patient and generates a health assessment which is described in further detailed below. The medical data suitable includes physiological data, laboratory data, respiratory data, and the like.

In one embodiment, the health assessment is displayed at the health assessment system 12. In another embodiment, the health assessment is an electronic file and saved in the IT infrastructure 10, such as in the patient information system 20. In some embodiments, health assessments are electronically messaged to clinicians using, for example, email and/or printed using, for example, a laser printer, an inkjet printer and so on. In a further embodiment, the health assessment is displayed on the one or more patient monitoring systems 14, one or more ventilation devices 16, one or more laboratory information systems 18, a patient information system 20, and the like. The health assessment system 12 also stores the health assessment into a health assessment database 30.

For example, the patient monitoring systems 14 obtain physiological data for patients (not shown) cared for by the medical institution. The physiological data suitably include data indicative of one or more physiological parameters, such as oxygen saturation, inspired oxygen, blood gas levels, ECG data, heart rate, respiratory data, temperature, blood oxygen saturation, level of consciousness, and so on. As to the former, sensors 32, such a pulse oximeter, electrocardiographic (ECG) electrodes, transcutaneous blood gas monitor, blood pressure sensors, and so on, measuring physiological parameters of patients can be employed. For example, the pulse oximeter indirectly measures the oxygen saturation of the patient's blood using infrared technology, called the SpO₂. The electrocardiograph (ECG) captures the patient's heart waveforms, and extracts from these waveforms the patient's heart rate and respiratory rate. The transcutaneous blood gas monitor non-invasively captures the patient's blood gas levels, including but not limited to oxygen (TcPO₂), carbon dioxide (TcPCO₂), and the like. It should also be appreciated that the patient monitoring system 14 including monitoring settings including, but not limited to, sampling rate, alarm settings, and the like. In one embodiment, the settings of the patient monitoring system 14 are automatically adjusted according to the health assessment of the patient and will described with further detail below. Further, the patient data can be generated automatically and/or manually. As to the latter, user input devices 34 can be employed. In some embodiments, the patient monitoring systems 14 include display devices 36 providing users a user interface within which to manually enter the patient data and/or for displaying generated patient data. The collected physiological data are concurrently transmitted to the patient information system 14 where the physiological data are displayed and stored.

Likewise, the ventilation devices 16 obtain respiratory data of a patient. The ventilation device 16 includes a continuous positive airway pressure (CPAP) device, a ventilator, an oxygen hood, nasal cannula, other oxygen delivery device, and the like. The ventilation device 16 has settings including, but not limited to, fraction of inspired oxygen (FiO₂), gas pressures applied to the patient (positive end expiratory pressure [PEEP] for ventilators, inspiratory pressure, and the like). In one embodiment, the settings of the ventilation device 16 are automatically adjusted according to the health assessment of the patient as described with further detail below. The respiratory data suitably include data indicative of one or more respiratory parameters, such as oxygen saturation, inspired oxygen, gas pressures, respiratory data, and so on. Further, the respiratory data can be generated automatically and/or manually. As to the latter, user input devices 38 can be employed. In some embodiments, the ventilation device 16 include display devices 42 providing users a user interface within which to manually enter the respiratory data and/or for displaying generated respiratory data. The collected respiratory data are concurrently transmitted to the patient information system 14 where the physiological data are displayed and stored.

Similarly, the laboratory information system 18 generates laboratory data from tests which are done on clinical specimens in order to get information relating to the health of a patient as pertaining to the diagnosis, treatment, and prevention of disease. The laboratory testing including atrial blood gases, anemia, hematological blood testing, coagulation laboratory testing, chemical blood, urine and body fluid testing, microbiology testing, urine laboratory testing, serological laboratory testing, cytology, histology, and pathology testing, immunohematology and blood banking testing, and the like. The laboratory information system 18 generates laboratory data reflecting the laboratory tests and stores the generated laboratory data to a laboratory database 46. In some embodiments, the laboratory information systems 18 include display devices 48 and a user interface 50 within which to manually enter the laboratory data and/or for displaying generated laboratory data to clinicians. The collected laboratory data are concurrently transmitted to the patient information system 14 where the laboratory data are displayed and stored. The health assessment system 12 acquires and displays requested laboratory data for the clinician to interpret.

The patient information system 20 stores physiological data, respiratory data, and laboratory data from the IT infrastructure 10, such as from the one or more patient monitoring systems 14, one or more ventilation devices 16, one or more laboratory information systems 18, in one or more databases 52 of the IT infrastructure 10. The patient information system 20 also stores health assessment generated by the health assessment system 12 in the one or more databases 52 of the IT infrastructure. The patient information system 20 further stores demographic patient information, including but not limited to patient's name, birth date, birth weight, gestational age, maternal information, delivery method, medications, and the like. It is also contemplated that the patient information system 20 stores physiological data, respiratory data, laboratory data, and health assessments generated from other IT infrastructures. In some embodiments, the patient information system 20 also stores physiological data, respiratory data, laboratory data, and medical reports generated from user input devices 54 in the database 52 and/or allows stored physiological data, respiratory data, laboratory data, and medical reports to be viewed on display devices 56. Examples of patient information systems include, but are not limited to, electronic medical record systems, departmental systems, and the like.

As mentioned above, the health assessment system 12 assesses the health of the patients (not shown) cared for by the medical institution. The health assessment system 12 analyzes the medical data received from the one or more patient monitoring systems 14, one or more ventilation devices 16, one or more laboratory information systems 18, and patient information system 20 and generates a health assessment of the patient. Specifically, the health assessment system 12 quantifies the recovery response of the patient to an oxygen desaturation with and without caregiver intervention. To quantify the recovery response and generate the health assessment, the health assessment system 12 determines a slope or time of the recovery response. Specifically, the health assessment system 12 analyzes a physiological or respiratory parameter, such as SpO₂. A high value of the physiological or respiratory parameter indicates a good health status of the patient and a low value indicates deteriorated health. The low values are detected by monitoring when the physiological or respiratory parameter crosses a predetermined low threshold. The time derivative of the physiological or respiratory parameter or slope after deterioration is then identified to assess the health of the patient. It should also be appreciated that the time interval between the physiological or respiratory parameter between a predetermined low threshold and recovering above a predetermined high threshold is also utilized to assess the health of the patient. In another embodiment, the physiological or respiratory parameter increase after a fixed amount of time after occurrence of the deterioration is utilized to assess the health of the patient.

For example, the SpO₂ waveform measured by the patient monitoring system 14 and/or ventilation device 16 is analyzed. SpO₂ waveform segments that indicate oxygen desaturations less than 85 percent are captured and stored for analysis. Information about changes in FiO₂ extracted from the patient monitoring system 14 and/or ventilation device 16 or as entered by the caregiver are utilized to determine whether there was a response by the caregiver to the patient's desaturation. A response to the patient's desaturation includes increasing the FiO₂ setting on the ventilation device, adjusting the position of the patient, and the like. In one embodiment, the health assessment system 12 establishes the presence of support by detecting the occurrence of a change in FiO₂, or a continuous high FiO₂ value, or by detection of nurse activity, e.g. by detecting the fact the incubator is open or the light is on. In the latter case, support may be given by manual stimulation of the baby. The health assessment system 12 then classifies the SpO₂ waveform segments as with or without caregiver response and compares the segments. The analysis further indicates whether the responses to desaturation are the same or different from those with or without caregiver response. When the responses are the same with or without caregiver response, and the patient recovers quickly, then the patient is considered to be in good health. When the responses are the same with or without caregiver response, and the patient does not recover quickly, then the patient is considered to be in poor health.

In another embodiment, the health assessment system 12 incorporates medical data received from the laboratory information system 18 and the medical information system 20. For example, information about medications that the patient is taking that affect respiratory responses are incorporated in the analysis of medical data to determine the health of the patient. In another embodiment, the health assessment system 12 incorporates laboratory results that indicate ventilation and oxygenation health. In a further embodiment, the health assessment system 12 incorporates active problems lists that provide information about the patient's current health status and comorbidities that may affect respiratory responses.

After analysis of the medical data, the health assessment system 12 generates and displays a health assessment to indicate to the caregiver an assessment of the patient's health status. In a further embodiment, the health assessment is displayed on the one or more patient monitoring systems 14, one or more ventilation devices 16, one or more laboratory information systems 18, a patient information system 20, and the like. In another embodiment, the settings of the patient monitoring system 14 and/or ventilation device 16 are adjusted in accordance with the health assessment. For example, if the patient is determined to be in poor health, the fraction of inspired oxygen (FiO₂) and/or gas pressures applied to the patient along with patient support may be increased. If the patient is determined to be in good health, the fraction of inspired oxygen (FiO₂), gas pressures applied to the patient, or patient support may be removed or decreased.

In another embodiment, the clinical decision support system (CDSS) 22 receives medical data from the IT infrastructure 10, such as from the one or more patient monitoring systems 14, one or more ventilation devices 16, one or more laboratory information systems 18, report medical system 12 and/or the patient information system 14 and assesses the health of the patients (not shown) cared for by the medical institution. The CDSS 22 provides clinicians with an assessment of a patient's health and generates a health assessment describing the patient's health status. After the medical data are acquired, the CDSS 22 assesses the health of the patient and generates a health assessment as described above. Specifically, the health assessment system 12 analyzes the medical data received from the one or more patient monitoring systems 14, one or more ventilation devices 16, one or more laboratory information systems 18, and patient information system 20 and generates a health assessment of the patient. After analysis of the medical data, the CDSS 22 generates and displays a health assessment to indicate to the caregiver an assessment of the patient's health status.

It should also be appreciated the present application can be generalized to other types of patient support that are manually adjusted, thus giving rise to a similar pattern of supported and unsupported recoveries. For example, comparison of bradycardia events in pediatric or adult patients in the Intensive Care Unit (ICU) may enable distinguishing stable and unstable bradycardia patients, which would require different clinical interventions. In another example, monitoring the adult, pediatric, or neonatal patient's respiratory rate for apnea events may indicate an improvement in the patient's condition if the patient experiences fewer apneas and recovers more quickly to apneas with or without intervention, or it may indicate clinical deterioration if the apnea events are more frequent and the patient takes longer to recover from the apnea event with or without intervention.

The components of the IT infrastructure 10 suitably include processors 64 executing computer executable instructions embodying the foregoing functionality, where the computer executable instructions are stored on memories 66 associated with the processors 64. It is, however, contemplated that at least some of the foregoing functionality can be implemented in hardware without the use of processors. For example, analog circuitry can be employed. Further, the components of the IT infrastructure 10 include communication units 68 providing the processors 64 an interface from which to communicate over the communications network 24. Even more, although the foregoing components of the IT infrastructure 10 were discretely described, it is to be appreciated that the components can be combined.

With reference to FIG. 2, a quantification of recovery through time derivation/slope is illustrated. A graph 100 includes an axis x 102 representing a value of a physiological/respiratory parameter and an axis t representing time 104. A line 106 illustrates the value of the physiological/respiratory parameter over time. The graph 100 also includes a low threshold 108, e.g. 85% oxygen desaturation, which indicates deteriorated health of the patient. As shown in the graph 100, at a point 110, the patient is experiencing deteriorated health, such as oxygen desaturation. A time derivative of the physiological/respiratory parameter or slope after the deterioration 112 is identified and utilized to assess the health of the patient. A steep slope (fast recovery) is indicative of good health. A flat slope (slow recovery) is indicative of weak health.

With reference to FIG. 3, another quantification of recovery through time interval is illustrated. A graph 200 includes an axis x 202 representing a value of a physiological/respiratory parameter and an axis t representing time 204. A line 106 illustrates the value of the physiological/respiratory parameter over time. The graph 200 also includes a low threshold 208 which indicates deteriorated health of the patient and a high threshold 210 which indicates a recovery of the patient. As shown in the graph 200, at a point 212, the patient is experiencing deteriorated health such as oxygen desaturation. At a point 214, the patient has recovered from the deteriorated health. The time interval dt 216 between the physiological/respiratory parameter going below the low threshold 208 and recovering the high threshold 210 is determined and utilized to assess the health of the patient. A short time interval indicates a strong recovery. Alternatively, an increased change in x in a fixed time interval can be used as an indicator for patient health; here, a large increased change in x indicates good health.

FIG. 4 illustrates several recovery and support scenarios and the recommendation for maintaining or withdrawing/reducing ventilation support 300. As shown in scenario 302, the patient has a fast recovery (steep slope/short recovery time dt/large increased change in x) with support and a slow recovery (flat slope/long recovery time dt/small increased change in x) without support indicating weak health. The recommendation for scenario 302 is to maintain the same support to the patient. In scenario 304, the patient has a slow recovery with support and a slow recovery without support indicating weak health. The recommendation for scenario 304 is to maintain the same support to the patient. In scenario 306, the patient has a fast recovery with support and a fast recovery without support indicating good health. The recommendation for scenario 306 is to withdraw or reduce the patient support.

| | SpO₂ delta after 80 seconds | | |
|---|---|---|---|
| | for SpO₂ dip of 30% | | |
| Patient | With support | Without support | Advice for support |
| 1 | 9% (slow) | 16% (fast) | maintain |
| 2 | 10% (slow) | 10% (slow) | maintain |
| 3 | 14% (fast) | 14% (fast) | withdraw/reduce |

For example, a recovery and support scenario includes monitoring the change in SpO₂ for 80 seconds after a dip of SpO₂ of 30% as shown in the table above. The 80-second interval is selected as this is in the range of the time delay between changing the FiO₂ setting and a physiological response in the SpO₂. However, it should be contemplated that other time intervals be utilized. Specifically, after a patient experiences a 30% dip or drop in SpO₂, the health assessment system monitors the change in SpO₂ for 80 seconds to assess the health of the patient. As shown, patient 1 has a 9% increase in SpO₂ (slow recovery) with support and a 16% increase in SpO₂ (fast recovery) without support over the 80 second interval, the recommendation for the scenario for patient 1 is to maintain the same support to the patient. Patient 2 has a 10% increase in SpO₂ (slow recovery) with support and a 10% increase in SpO₂ (slow recovery) without support over the 80 second interval, the recommendation for the scenario for patient 2 is to maintain the same support to the patient. Patient 3 has a 14% increase in SpO₂ (fast recovery) with support and a 14% increase in SpO₂ (fast recovery) without support over the 80 second interval, the recommendation for the scenario for patient 3 is to withdraw or reduce the support to the patient.

With reference to FIG. 5, an exemplary illustration of a response to an apnea or other oxygen desaturation event is illustrated. A graph 400 illustrates includes a SpO₂ delta axis 402 representing the change in the SpO₂ and a SpO₂ dip axis 404 representing the dip or drop in the SpO₂. The graph 400 further includes indicators indicating the responses 406 to a SpO₂ dip with and without response. The graph also includes an indicator of the average responses to a SpO₂ dip with 408 and without response 410. As shown in the graph 400, the patient has a fast recovery with support and a slow recovery without support and thus the patient's support should be maintained.

FIG. 6 illustrates another exemplary illustration of a response to an apnea or other oxygen desaturation event. A graph 500 includes a SpO₂ delta axis 502 representing the change in the SpO₂ and a SpO₂ dip axis 504 representing the dip of the SpO₂. The graph 500 further includes indicators indicating the responses 506 to a SpO₂ dip with and without response. The graph also includes an indicator of the average responses to a SpO₂ dip with 508 and without response 510. As shown in the graph 500, the patient has a slow recovery with support and a slow recovery without support and thus the patient's support should be maintained.

With reference to FIG. 7, further exemplary illustration of a response to an apnea or other oxygen desaturation event is illustrated. A graph 600 illustrates includes a SpO₂ delta axis 602 representing the change in the SpO₂ and a SpO₂ dip axis 604 representing the dip of the SpO₂. The graph 600 further includes indicators indicating the responses 606 to a SpO₂ dip with and without response. The graph also includes an indicator of the average responses to a SpO₂ dip with 608 and without response 610. As shown in the graph 600, the patient has a fast recovery with support and a fast recovery without support and thus the patient's support can be withdrawn or reduced.

With reference to FIG. 8, a method 700 for assessing a patient's health is illustrated. In a step 702, medical data are received representing a patient's oxygen saturation. In a step 704, an oxygen desaturation event is detected from the medical data. A recovery response from the oxygen desaturation event with caregiver intervention is determined in a step 706. In a step 708, a recovery response from the oxygen desaturation event is determined without caregiver intervention is determined. It should be appreciated that the oxygen desaturation events are not planned and occur spontaneously and at random times. For example, a recovery response from the oxygen desaturation event determined without caregiver intervention can occur before a recovery response from the oxygen desaturation event is determined with caregiver intervention. A health assessment is generated from the recovery responses with and without caregiver intervention in a step 710. In a step 712, the health assessment is displayed.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), and the like; a user input device includes one or more of a mouse, a keyboard, a touch screen display, one or more buttons, one or more switches, one or more toggles, and the like; and a display device includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A health assessment system for assessing the health of a patient, said system being adapted to:
receive medical data representing a patient's oxygen saturation;
detect an oxygen desaturation event from the medical data;
determine a recovery response following the oxygen desaturation event; and
generate a health assessment based on the patient's recovery response, wherein the health assessment is based on the patient's recovery response with and without caregiver intervention.

2. (canceled)

3. The system according to claim 1, wherein caregiver intervention is detected by at least one of a change in an inspired oxygen level and a detection of caregiver activity.

4. The system according to claim 1, wherein the recovery response is determined based on a recovery time of the patient after the oxygenation desaturation event is detected.

5. The system according to claims 1, wherein the recovery response is determined based on a time period from a detection of the oxygenation desaturation event to the patient recovering to predetermined oxygen saturation.

6. The system according to claim 1, wherein the recovery response is based on a rate of recovery after detection of the oxygen desaturation event.

7. The system according to claim 1, wherein the recovery response is based on an increase in oxygen saturation after a fixed amount of time after detection of the oxygen desaturation event.

8. (canceled)

9. The system according to claim 1 wherein at least one of the settings of a ventilator and patient treatment are adjusted in response to the health assessment.

10. (canceled)

11. (canceled)

12. (canceled)

13. A method performed by one or more processors for assessing the health of a patient, the method comprising:
receiving medical data representing a patient's oxygen saturation;
detecting an oxygen desaturation event from the medical data;
determining a recovery response from the oxygen desaturation event; and
generating a health assessment based on the patient's recovery response, wherein the health assessment is based on the patient's recovery response with and without caregiver intervention.

14. (canceled)

15. (canceled)

16. The method according to claim 13, wherein caregiver intervention is detected by at least one of a change in a ventilator setting and a detection of caregiver activity.

17. The method according to claim 13,
wherein determining the recovery response includes determining a measure of the recovery time of the patient after the oxygenation desaturation event is detected.

18. The method according to claim 13, wherein determining the recovery response includes at least one of:
determining a time period from a detection of the oxygenation desaturation event to the patient reaching predetermined oxygen saturation,
determining an increase in the oxygen saturation after a fixed amount of time after detection of the oxygenation desaturation event, and
determining a rate of an increase in the oxygen saturation.

19. (canceled)

20. A non-transitory computer readable medium
carrying software which controls one or more processors to perform the method according to claim 18.

21. The method according to claim 13, the method further comprising:
displaying at least one of the determined recovery response and the health assessment based on the determined recovery response,

22. The system according to claim 1, said system being further adapted to:
display at least one of an indicator of the recovery response and the health assessment from the recovery response.

23. A system for assessing the health of a patient, said system, comprising:
one or more sensors for generating the medical data representing a patient's oxygen saturation;
the health assessment system, according to claim 1.
